(19) United States
(12) Patent Application Publication
(10) Pub. No.: US 2026/0224601 A1
(43) Pub. Date: Aug. 6, 2026

(54) TOPICAL FORMULATION FOR TREATING ATOPIC DERMATITIS, AND PREPARATION METHOD AND USE THEREOF

(71) Applicant: SHANGHAI SKIN DISEASE HOSPITAL, Shanghai (CN)

(72) Inventors: Xin MA, Shanghai (CN); Le KUAI, Shanghai (CN); Mi ZHOU, Shanghai (CN); Qingkai LIU, Shanghai (CN); Ying LUO, Shanghai (CN); Hang ZHAO, Shanghai (CN); Guoshu DENG, Shanghai (CN); Chunjie GAO, Shanghai (CN); Ding LI, Shanghai (CN); Yue JIANG, Shanghai (CN); Yi ZHANG, Shanghai (CN); Tianyou CHEN, Shanghai (CN); Yiqi GONG, Shanghai (CN); Zhan ZHANG, Shanghai (CN); Jiankun SONG, Shanghai (CN); Yue LUO, Shanghai (CN); Xiaoya FEI, Shanghai (CN); Zhongjian CHEN, Shanghai (CN); Quangang ZHU, Shanghai (CN); Zongguang TAI, Shanghai (CN); Tianyou MA, Shanghai (CN); Ruiping WANG, Shanghai (CN); Bin LI, Shanghai (CN)

(73) Assignee: SHANGHAI SKIN DISEASE HOSPITAL, Shanghai (CN)

(21) Appl. No.: 19/422,672

(22) Filed: Dec. 17, 2025

Related U.S. Application Data
(63) Continuation-in-part of application No. PCT/CN2025/099255, filed on Jun. 5, 2025.

(30) Foreign Application Priority Data
Feb. 6, 2025 (CN) 202510131320.9

(51) Int. Cl. A61K 31/7048; A61K 9/00; A61K 9/06; A61K 47/10; A61K 47/14; A61P 17/00

(52) U.S. Cl. CPC A61K 31/7048; A61K 9/0014; A61K 9/06; A61K 47/10; A61K 47/14; A61P 17/00

(57) ABSTRACT

A topical formulation for treating atopic dermatitis (AD), and a preparation method and use thereof, belonging to the technical field of development of traditional Chinese medicine (TCM) topical formulations. An active ingredient of the topical formulation for treating AD is neoliquiritin with a concentration of 0.5 wt % to 1.5 wt %.

### CROSS REFERENCE TO RELATED APPLICATION

The present application is a continuation-in-part of International Patent Application No. PCT/CN2025/099255, filed on Jun. 5, 2025, which claims priority to Chinese Patent Application No. 202510131320.9 filed with the China National Intellectual Property Administration (CNIPA) on Feb. 6, 2025 and entitled “TOPICAL FORMULATION FOR TREATING ATOPIC DERMATITIS, AND PREPARATION METHOD AND USE THEREOF”, which are incorporated herein by reference in their entirety.

### REFERENCE TO SEQUENCE LISTING

A computer readable XML file entitled “Sequence Listing”, that was created on Nov. 24, 2025, with a file size of 8,005 bytes, contains the sequence listing for this application, has been filed with this application, and is hereby incorporated by reference in its entirety.

### TECHNICAL FIELD

The present disclosure belongs to the technical field of development of traditional Chinese medicine (TCM) topical formulations, and relates to a topical formulation for treating atopic dermatitis (AD), and a preparation method and use thereof.

### BACKGROUND

Atopic dermatitis (AD) is a chronic, relapsing, inflammatory skin disease characterized most fundamentally by dry skin, chronic eczema-like dermatitis, and intense pruritus. Since patients generally concurrently suffer from other atopic conditions such as allergic rhinitis and asthma, and approximately 40% to 80% have a family history of allergies, AD is considered a systemic disease. The pathogenesis of AD is influenced by multiple factors and is the result of the interaction of various factors such as genetics, immunity, infectious, environment. Current treatments for AD primarily involve topical corticosteroids, topical calcineurin inhibitors, oral antihistamines, systemic immunosuppressants, and systemic corticosteroids. While these drugs demonstrate desirable short-term therapeutic efficacy against AD, their long-term use is associated with significant side effects and a high recurrence rate. For example, corticosteroids are first-line drugs for AD and are always the preferred treatment. However, long-term topical use of the corticosteroids may lead to side effects such as skin atrophy, telangiectasia, striae atrophy, purpura, and hypertrichosis.

TCM topical formulations have shown certain advantages in improving clinical symptoms and reducing recurrence rates in patients with mild to severe AD. In a randomized, double-blind clinical trial, Indigo Naturalis ointment has effectively reduced the severity of skin lesions and pruritus while maintaining a favorable safety profile. Multiple studies have demonstrated that various TCM topical formulations ameliorate skin lesion conditions in AD mouse models and reduce inflammatory markers in both serum and skin lesions. Licorice is known for its ability to clear heat, detoxify, and alleviate acute and severe pain. Consequently, it exhibits a wide range of pharmacological actions, including anti-inflammatory, antitumor, antibacterial, and immunomodulatory activities, making it widely used in dermatological conditions. The primary active component of licorice, liquiritin, has been demonstrated to possess multiple pharmacological activities such as anti-inflammatory, antioxidant, and anti-apoptotic effects, and is therefore used in the treatment of various skin diseases like photoaging and psoriasis. Neoliquiritin, as another active constituent of licorice, has also been proven to exhibit potent anti-inflammatory and antiviral effects.

### SUMMARY

In view of this, an objective of the present disclosure is to provide a topical formulation for treating atopic dermatitis (AD), and a preparation method and use thereof. In the present disclosure, neoliquiritin is used as an active ingredient to treat AD, with significant effects, thereby expanding novel application areas for the neoliquiritin.

To solve the above technical problem, the present disclosure provides the following technical solutions:

The present disclosure provides a topical formulation for treating AD, where an active ingredient of the topical formulation is neoliquiritin.

The present disclosure further provides a cream for treating AD, including the following components by mass concentration: 0.5% to 1.5% of neoliquiritin, 4% to 13% of a solvent, 5% to 25% of an oil phase, 56% to 87% of an aqueous phase, 2% to 8% of an emulsifier, and 0.05% to 0.15% of a preservative.

The present disclosure further provides a method for preparing the cream, including the following steps: (1) weighing and mixing the oil phase, the emulsifier, and the preservative to obtain a first mixture, weighing the aqueous phase to obtain a second mixture, and dissolving the neoliquiritin with the solvent to obtain a third mixture; (2) mixing the first mixture and the second mixture under heating, homogenizing, adding the third mixture and homogenizing again; and (3) cooling an obtained twice-homogenized product to obtain the cream.

The present disclosure further provides use of the topical formulation in manufacture of drugs for treating AD and in manufacture of drugs for inhibiting epidermal proliferation and/or dermal lymphocyte infiltration.

### BRIEF DESCRIPTION OF THE DRAWINGS

FIGS. 1A-1C show the effect of neoliquiritin solution on HaCaT cell viability; where FIG. 1A shows the chemical structural formula of neoliquiritin; FIG. 1B shows the effect of different concentrations of neoliquiritin solution on the proliferative activity of HaCaT cells; FIG. 1C shows the effect of 2 μM and 10 μM neoliquiritin solutions on the mRNA expression of inflammatory factors IL-4 and IL-13 in HaCaT cells.

FIGS. 2A-2C show the skin lesion conditions and dermatitis scores of different treatment groups in the mouse model of AD; where FIG. 2A shows typical photographs of auricle skin lesions and lesion changes in mice from different treatment groups (Day 12) (n=5); FIG. 2B shows the auricular thickness of mice in different treatment groups (n=5); FIG. 2C shows the eczema area and severity index (EASI) scores of skin lesions in mice from different treatment groups (n=5).

FIGS. 3A-3C show the histopathological conditions of different treatment groups in the mouse model of AD; where FIG. 3A shows the histopathological changes in mouse auricles from different treatment groups (×100) (n=5); FIG. 3B shows the epidermal thickness of mouse auricles in different treatment groups (n=5); FIG. 3C shows the quantitative comparison of dermal lymphocyte counts in mice from different treatment groups (n=5).

FIG. 4 shows the pathological changes in the liver, spleen, and kidney of mice after topical administration of neoliquiritin (×200).

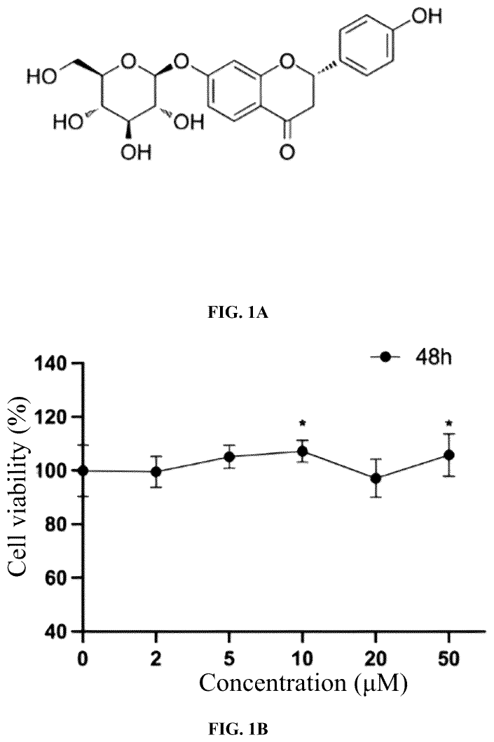

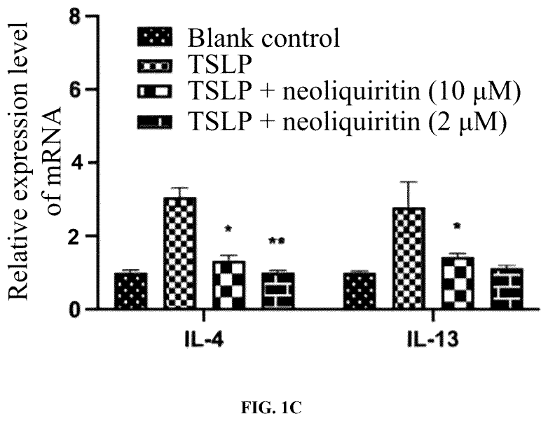

NOTE: in FIG. 1A to FIGS. 3C, compared with the model group, **p<0.01, ***p<0.001; compared with the normal control group, ##p<0.01, #p<0.001.

### DETAILED DESCRIPTION OF THE EMBODIMENTS

The present disclosure provides a topical formulation for treating AD, where an active ingredient of the topical formulation is neoliquiritin.

Neoliquiritin is a flavonoid compound, commonly found in the medicinal parts of the leguminous plant, licorice (Glycyrrhiza uralensis), possessing various pharmacological activities such as anti-inflammatory and antiviral effects. Its chemical structural formula is shown in FIG. 1A, with the molecular formula of C₂₁H₂₂O₉, the molecular weight of 418.4, and the CAS number of 5088-75-5.

In the present disclosure, a concentration of the neoliquiritin is preferably 0.5 wt % to 1.5 wt %, more preferably 0.8 wt % to 1.5 wt %, and most preferably 1 wt %.

In the present disclosure, a preferred dosage form of the topical formulation includes a patch, a paste, an ointment, a cream, a gel, an oil, a microneedle preparation, a coating agent, a cataplasm, a spray, and a dressing. It is preferred that the topical formulation further includes a pharmaceutically acceptable auxiliary material. There are no specific limitations on the auxiliary materials or the preparation methods for the topical formulation, any commonly used auxiliary materials and conventional preparation methods in the pharmaceutical field are acceptable.

The present disclosure further provides a cream for treating AD, including the following components by mass concentration: 0.5% to 1.5% of neoliquiritin, 4% to 13% of a solvent, 5% to 25% of an oil phase, 56% to 87% of an aqueous phase, 2% to 8% of an emulsifier, and 0.05% to 0.15% of a preservative; preferably the following components by mass concentration: 0.8% to 1.2% of the neoliquiritin, 6% to 10% of the solvent, 10% to 20% of the oil phase, 65% to 75% of the aqueous phase, 3% to 5% of the emulsifier, and 0.08% to 0.12% of the preservative; more preferably the following components by mass concentration: 1% of the neoliquiritin, 8.3% of the solvent, 14.0% of the oil phase, 71.9% of the aqueous phase, 4.7% of the emulsifier, and 0.1% of the preservative.

In the present disclosure, the solvent is preferably one or more selected from the group consisting of diethylene glycol monoethyl ether, ethanol, and propylene glycol, more preferably diethylene glycol monoethyl ether.

The oil phase is preferably one or more selected from the group consisting of glyceryl mono- and distearate, cetostearyl alcohol, liquid paraffin, vaseline, and lanolin, more preferably glyceryl mono- and distearate, cetostearyl alcohol, and liquid paraffin, and the mass ratio of the glyceryl mono- and distearate, the cetostearyl alcohol, and the liquid paraffin is 3:5:6.

The aqueous phase is preferably one or more selected from the group consisting of purified water, deionized water, and glycerol, more preferably purified water and glycerol, and the mass ratio of the purified water and the glycerol is 9.3:1.

The emulsifier is preferably one or more selected from the group consisting of polyethylene glycol-7-stearate, polyoxyethylene (21) stearyl ether, polyoxyethylene (2) stearyl ether, polyglyceryl oleate, and sodium lauryl sulfate, more preferably the polyethylene glycol-7-stearate, the polyoxyethylene (21) stearyl ether, the polyoxyethylene (2) stearyl ether, and the polyglyceryl oleate, and the mass ratio of the polyethylene glycol-7-stearate, the polyoxyethylene (21) stearyl ether, the polyoxyethylene (2) stearyl ether, and the polyglyceryl oleate is 4:9:9:6.

The preservative is preferably one or more selected from the group consisting of ethylparaben, methylparaben, and phenoxyethanol, more preferably ethylparaben.

The present disclosure further provides a method for preparing the cream for treating AD, including the following steps:
(1) weighing and mixing the oil phase, the emulsifier, and the preservative to obtain a first mixture, weighing the aqueous phase to obtain a second mixture, and dissolving the neoliquiritin with the solvent to obtain a third mixture;
(2) mixing the first mixture and the second mixture under heating, homogenizing, adding the third mixture and homogenizing again; preferably, heating the first mixture and the second mixture to 85° C. before the mixing, homogenizing for 3 min, cooling the obtained homogenized product to 60° C., adding with the third mixture, and then homogenizing again for 3 min; and
(3) cooling an obtained twice-homogenized product to obtain the cream; preferably cooling to 40° C., and packaging the cream.

The present disclosure further provides use of the topical formulation in manufacture of drugs for treating AD and in manufacture of drugs for inhibiting epidermal proliferation and/or dermal lymphocyte infiltration.

The technical scheme of the present disclosure has the following beneficial effects:

By utilizing neoliquiritin as the active ingredient in the topical formulation for treating AD, the present disclosure provides a novel therapeutic option for AD, demonstrating significant advantages in multiple aspects.

Regarding safety, neoliquiritin has no adverse effects on the normal growth of skin cells and exhibits a desirable safety profile, providing an essential prerequisite for its topical administration. Furthermore, topical administration shows no significant toxic or side effects on vital organs. Compared to traditional therapeutic drugs like corticosteroids, which may lead to side effects including skin atrophy, telangiectasia, striae atrophy, purpura, and hypertrichosis from long-term topical administration, neoliquiritin offers a superior safety profile, thereby reducing the risk of adverse drug reactions for patients.

In terms of therapeutic efficacy, neoliquiritin effectively intervenes in the inflammatory response process of AD at the cellular level. It modulates immune-related inflammatory signaling pathways from the source, laying the foundation for alleviating skin inflammation. Neoliquiritin shows a marked improvement effect on the skin symptoms of AD, effectively reducing manifestations such as skin inflammation, erythema, edema, and scaling, thereby enhancing the overall health of the skin. Additionally, neoliquiritin effectively regulates the pathological structure of skin tissue, promoting the repair and normalization of skin tissue, which in turn strengthens both the skin barrier function and immune homeostasis.

The formulation design of the neoliquiritin cream in the present disclosure ensures the stability, efficacy, and safety of the formulation enabling the drug to exert its therapeutic effects more effectively, improving drug bioavailability and duration of efficacy, and providing a reliable formulation basis for clinical application.

The present disclosure is further described below with reference to the accompanying drawings and examples.

Methods used in the following examples are conventional methods unless otherwise specified.

### Example 1

A topical cream for treating AD, prepared as follows:
(1) the following materials were weighed as the component A: 36 g of glyceryl mono- and distearate, 60 g of cetostearyl alcohol, 72 g of liquid paraffin, 1.2 g of ethylparaben, 8 g of polyethylene glycol-7-stearate, 18 g of polyoxyethylene (21) stearyl ether, 18 g of polyoxyethylene (2) stearyl ether, and 12 g of polyglyceryl oleate;
(2) 1,978.8 g of purified water and 84 g of glycerol were weighed as the component B;
(3) 100 g of diethylene glycol monoethyl ether was weighed and 12 g of neoliquiritin was added thereto, followed by ultrasonication for 30 min to fully dissolve as the component C;
(4) the component A and the component B were heated to 85° C., fully mixed under stirring, and homogenized for 3 min;
(5) when the mixture of the components A and B was cooled to approximately 60° C., the component C was added, and homogenization was continued for another 3 min; and
(6) when the resulting sample was cooled to approximately 40° C., it was packaged.

### Example 2

A topical cream for treating AD, prepared as follows:
(1) the following materials were weighed as the component A: 36 g of glyceryl mono- and distearate, 60 g of cetostearyl alcohol, 72 g of liquid paraffin, 1.2 g of ethylparaben, 8 g of polyethylene glycol-7-stearate, 18 g of polyoxyethylene (21) stearyl ether, 18 g of polyoxyethylene (2) stearyl ether, and 12 g of polyglyceryl oleate;
(2) 778.8 g of purified water and 84 g of glycerol were weighed as the component B;
(3) 100 g of diethylene glycol monoethyl ether was weighed and 12 g of neoliquiritin was added thereto, followed by ultrasonication for 30 min to fully dissolve as the component C;
(4) the component A and the component B were heated to 85° C., fully mixed under stirring, and homogenized for 3 min;
(5) when the mixture of the components A and B was cooled to approximately 60° C., the component C was added, and homogenization was continued for another 3 min; and
(6) when the resulting sample was cooled to approximately 40° C., it was packaged.

### Example 3

A topical cream for treating AD, prepared as follows:
(1) the following materials were weighed as the component A: 36 g of glyceryl mono- and distearate, 60 g of cetostearyl alcohol, 72 g of liquid paraffin, 1.2 g of ethylparaben, 8 g of polyethylene glycol-7-stearate, 18 g of polyoxyethylene (21) stearyl ether, 18 g of polyoxyethylene (2) stearyl ether, and 12 g of polyglyceryl oleate;
(2) 378.8 g of purified water and 84 g of glycerol were weighed as the component B;
(3) 100 g of diethylene glycol monoethyl ether was weighed and 12 g of neoliquiritin was added thereto, followed by ultrasonication for 30 min to fully dissolve as the component C;
(4) the component A and the component B were heated to 85° C., fully mixed under stirring, and homogenized for 3 min;
(5) when the mixture of the components A and B was cooled to approximately 60° C., the component C was added, and homogenization was continued for another 3 min; and
(6) when the resulting sample was cooled to approximately 40° C., it was packaged.

### Example 4

### HaCaT Cell Activity Assay

20 mg of neoliquiritin was weighed, dissolved in PBS via ultrasonication, and prepared into a stock solution with a final concentration of 50.00 mM based on the minimum solubility in DMSO. The stock solution was stored in a refrigerator at 4° C. for subsequent experiments.

Cell culture and treatment: HaCaT cells (purchased from Cell Lines Service (Eppelheim, 300493)) were cultured in DMEM medium containing 10% fetal bovine serum. The above cell lines were maintained in an incubator at 37° C. with 5% CO₂, routinely passaged for 3 generations, and then used for subsequent experiments.

### (1) Detection of HaCaT Cell Viability

The experimental groups for the neoliquiritin solution included a blank control group and different concentration groups (2, 5, 10, 20, and 50 μM), with 4 replicate wells set for each group. HaCaT cells were seeded into 96-well plates. When the cell confluence reached over 80%, the original medium was aspirated and discarded. Then, 0.1 mL of medium containing different drug concentrations was added to each well, while the blank control group received drug-free complete medium. The plates were subsequently returned to the incubator for continued cultivation. After 48 h of neoliquiritin treatment, the original medium was aspirated and discarded. The cells were washed once with 100 μL/well of PBS buffer, followed by the addition of 110 μL/well of CCK-8 working solution (where the CCK-8 working solution was prepared at a ratio of CCK8 reagent to complete cell culture medium of 1:10). The plates were then placed back into the incubator and incubated for another 2 h. Subsequently, the 96-well plates were placed in a multifunctional microplate reader, and the absorbance was measured at a wavelength of 450 nm. Cell viabilities were calculated based on the absorbance values.

The results were shown in FIG. 1B. The CCK-8 assay was used to screen the effective concentration of neoliquiritin in vitro by detecting its effect on HaCaT cell proliferative activity at different concentrations, thereby guiding subsequent experiments. Specifically, after 48 h of treatment with neoliquiritin solutions at 2, 5, 10, 20, and 50 μM, the cell proliferation rates showed no significant difference compared to the control group. This indicated that neoliquiritin solutions at concentrations ranging from 2 to 50 μM did not inhibit HaCaT cell proliferation, while also suggesting the favorable safety profile of this monomeric component.
(2) RT-qPCR Analysis of Inflammatory Factors IL-4 and IL-13 mRNAin TSLP-Stimulated HaCaT Cells Treated with 2 μM and 10 μM Neoliquiritin Solutions

Cells were cultured in 6-well plates and divided into a blank control group (NC) and treatment groups with different drug concentrations (0, 2, 10 PM), with 4 replicate wells set for each group. When HaCaT cells reached approximately 70% confluence, human thymic stromal lymphopoietin (TSLP) was added to the complete medium at a predetermined concentration (100 ng/mL) to simulate the inflammatory environment of AD. Then, the cells were stimulated for 48 h by adding 2 mL of TSLP and neoliquiritin solutions at the specified concentrations to the medium.

Total mRNA was extracted from HaCaT cells using the Trizol method (Beyotime, Shanghai, China). The concentration and purity of the RNA were determined by measuring the absorbance at 260/280 ratio using a UV spectrophotometer. cDNA was prepared using a reverse transcription kit, and reverse transcription was performed using reverse transcriptase. Relative quantitative data were analyzed using the 2^{-ΔΔCT} method. The primers used for polymerase chain reaction were listed in Table 1.

TABLE 1
Primers for PCR

| | Primers and sequences | | | |
|---|---|---|---|---|
| Gene | | SEQ ID | | SEQ ID |
| Name | Forward primer | NO: | Reverse primer | NO: |
| ACTB | CACCATTGGCAATGAGCGGTTC | 1 | AGGTCTTTGCGGATGTCCACGT | 2 |
| IL4 | CCGTAACAGACATCTTTGCTGCC | 3 | GAGTGTCCTTCTCATGGTGGCT | 4 |
| IL3 | ACGGTCATTGCTCTCACTTGCC | 5 | CTGTCAGGTTGATGCTCCATACC | 6 |
| TSLP | TATCTGGTGCCCAGGCTATTCG | 7 | TGAAGCGACGCCACAATCCTTG | 8 |

The results were shown in FIG. 1C. The mRNA expression levels of inflammatory factors IL-4 and IL-13 in HaCaT cells decreased at both 2 μM and 10 μM concentrations, with the most significant reduction observed at 10 μM (compared to the TSLP group, *p<0.05, **p<0.01).

### Example 5

### AD Mouse Model Experiment

1. Drug Preparation: drugs were prepared according to the methods in Examples 1 to 3.

### 2. Experimental Methods

### 2.1. Grouping and Intervention

Male C57BL/6 mice aged 6-8 weeks and weighing 20 g+2 g were randomly divided into 6 groups, with 5 mice in each group. The mice were housed in a temperature-controlled (20° C. to 26° C.) specific pathogen-free environment and provided with standard food and water. The grouping and treatment regimens were as follows:
(1) Normal control group: the inner and outer surfaces of the mouse auricles were smeared with 20 μL of absolute ethanol every day for 5 days, then stopped for 2 days, and then smeared for 5 days.
(2) Model group: the inner and outer surfaces of the mouse auricles were smeared with 20 μL of an ethanol solution containing 2 nmol of calcipotriol (MC903) every day for 5 days, then stopped for 2 days, and then smeared for 5 days.
(3) Treatment group with 0.5 wt % neoliquiritin: the inner and outer surfaces of the mouse auricles were smeared with 20 μL of an ethanol solution containing 2 nmol of calcipotriol (MC903) every day for 5 days, then stopped for 2 days, and then smeared for 5 days. After 6 h, the inner and outer surfaces of the mouse auricles were smeared with 5 mg each mouse auricle of the 0.5% neoliquiritin cream prepared in Example 1. The total treatment period was 12 days.
(4) Treatment group with 1.0 wt % neoliquiritin: the inner and outer surfaces of the mouse auricles were smeared with 20 μL of an ethanol solution containing 2 nmol of calcipotriol (MC903) every day for 5 days, then stopped for 2 days, and then smeared for 5 days. After 6 h, the inner and outer surfaces of the mouse auricles were smeared with 5 mg each mouse auricle of the 1.0% neoliquiritin cream prepared in Example 2. The total treatment period was 12 days.
(5) Treatment group with 1.5 wt % neoliquiritin: the inner and outer surfaces of the mouse auricles were smeared with 20 μL of an ethanol solution containing 2 nmol of calcipotriol (MC903) every day for 5 days, then stopped for 2 days, and then smeared for 5 days. After 6 h, the inner and outer surfaces of the mouse auricles were smeared with 5 mg each mouse auricle of the 1.5% neoliquiritin cream prepared in Example 3. The total treatment period was 12 days.
(6) Positive control group: the inner and outer surfaces of the mouse auricles were smeared with 20 μL of an ethanol solution containing 2 nmol of calcipotriol (MC903) every day for 5 days, then stopped for 2 days, and then smeared for 5 days. After 6 h, the inner and outer surfaces of the mouse auricles were smeared with 5 mg each mouse auricle of the 0.1% Desonide Cream. The total treatment period was 12 days.

All treatments started from the day of calcipotriol administration (Day 1). All animal experiments were approved by the Ethics Committee of Yueyang Hospital of Integrated Traditional Chinese and Western Medicine affiliated with Shanghai University of Traditional Chinese Medicine (TCM).

### 2.2. Observation Indicators

On days 1, 5, 8, and 12, the appearance of the mouse ears was photographed, and the skin thickness of the same part of the ear was measured with a vernier caliper and recorded. The severity of auricular skin inflammation was scored according to the eczema area and severity index (EASI) scoring criteria for AD, including four parameters: erythema, edema or exudation, scaling, and lichenification. The scores indicated severity: 0 (none), 1 (mild), 2 (moderate), and 3 (severe).

### 3. Histopathology

After recording the above data on day 12, the mice were euthanized, and 1×1 cm skin tissues were collected from both ears of each group. The tissues were fixed in 4% formalin solution for 48 h, embedded in paraffin, sectioned, and stained with hematoxylin-eosin (H&E). Four high-power fields were randomly selected from each section to measure epidermal thickness, and the average value was calculated.

### 4. Statistical Methods

Experimental data were analyzed using GraphPad Prism 9. All data were expressed as mean±standard deviation (SD). Multiple comparisons between groups were conducted using one-way analysis of variance (ANOVA) and Tukey's post-hoc test. p value<0.05 was considered statistically significant.

### 5. Experimental Results

### 5.1. Neoliquiritin in Improving Skin Lesions and Dermatitis Scores in the Calcipotriol-Induced AD Mouse Model

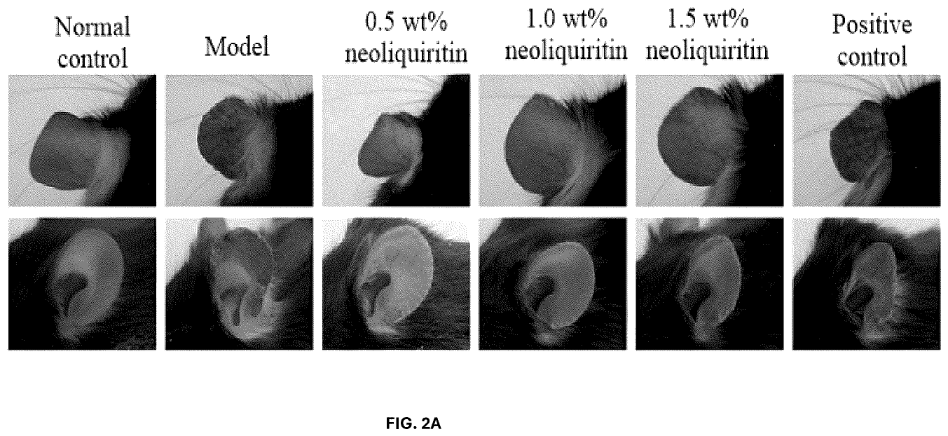

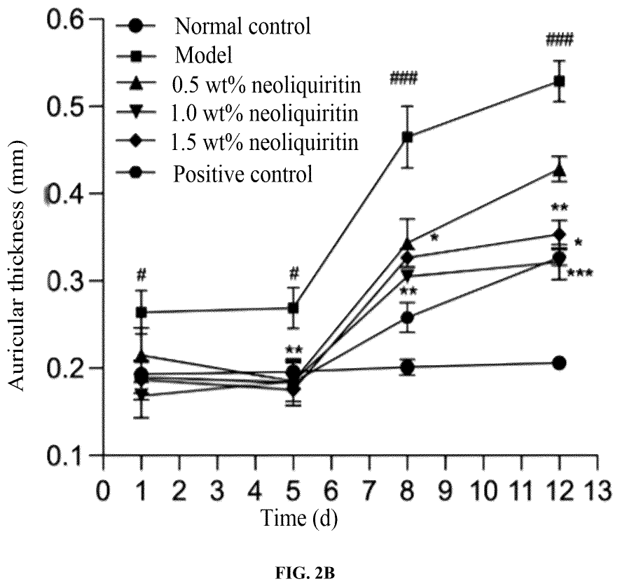

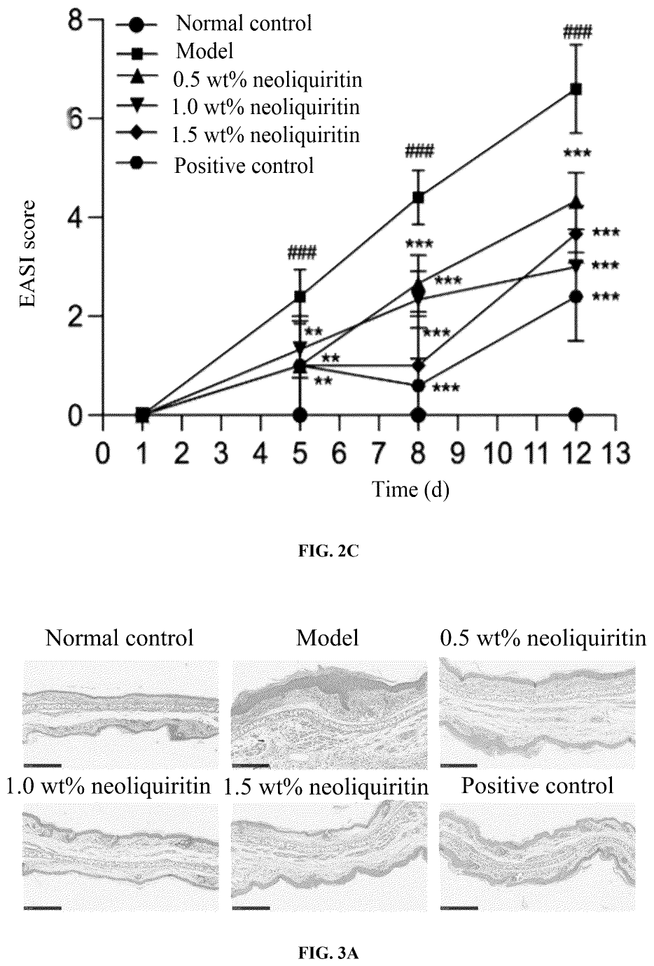

Typical photographs and conditions of auricular skin lesions in each group of mice were shown in FIG. 2A. The study found that the auricular skin thickness of calcipotriol-induced mice (model group) was significantly increased (^{###}p<0.001). The treatment group with 1.0 wt % neoliquiritin and the treatment group with 1.5 wt % neoliquiritin showed significant reductions in ear thickness compared to the MC903 group (*p<0.05, **p<0.01) (FIG. 2B). Compared to the MC903 group, the treatment group with 0.5 wt % neoliquiritin showed no significant difference in auricular skin thickness. In terms of EASI scores, the model group showed a significant increase compared to the normal control group (^{###}p<0.001), while the treatment group with three concentrations of neoliquiritin showed significant decreases compared to the model group (**p<0.01, ***p<0.001) (FIG. 2C). The above results indicated that both 1.0 wt % neoliquiritin and 1.5 wt % neoliquiritin could improve the appearance of skin lesions and auricular thickness in AD-like mice, with the treatment group with 1.0 wt % neoliquiritin showing better efficacy than the treatment group with 1.5 wt % neoliquiritin.

### 5.2. Neoliquiritin in Improving Histopathological Conditions in the Calcipotriol-Induced AD Mouse Model

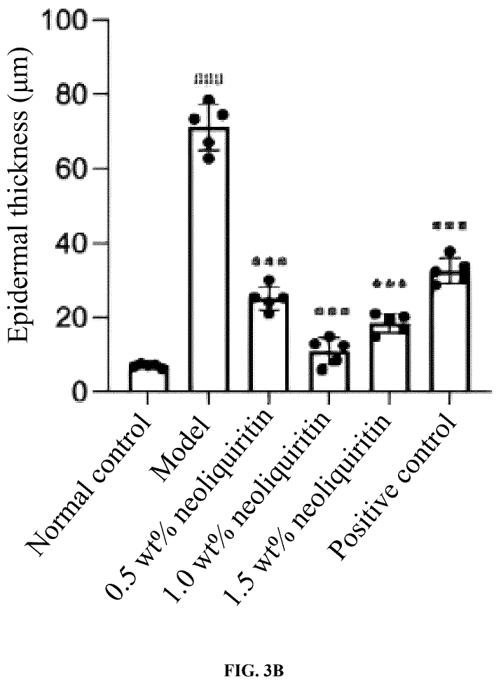

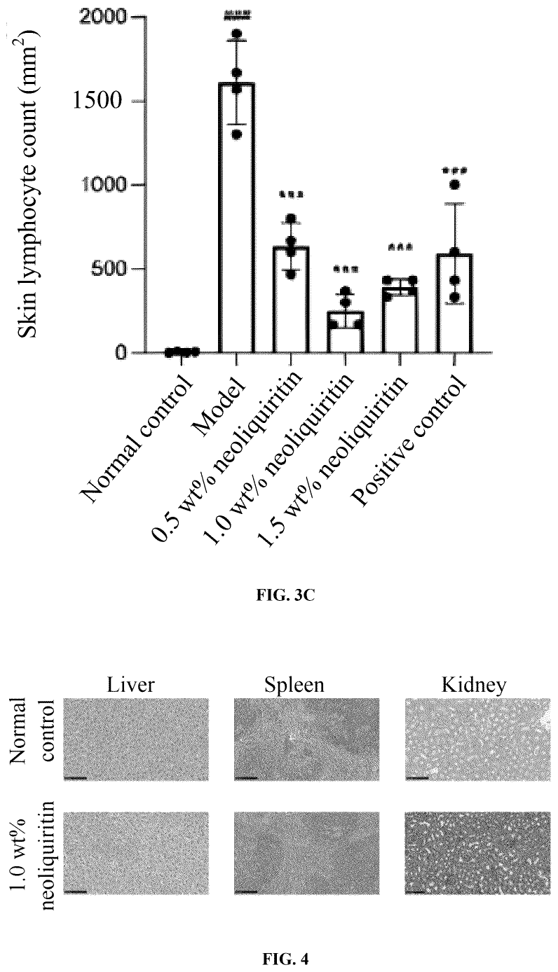

Based on the above results, the treatment group with three concentrations of neoliquiritin were selected for further study. The results showed that on day 12, the skin lesions of mice in the treatment group with three neoliquiritin concentrations were significantly improved compared to the model group (FIGS. 3A-3C). Histopathological sections of each group (FIG. 3A, scale bar=250 μm) showed epidermal hyperplasia, inflammatory cell infiltration in the dermis, and acanthosis in the model group. The treatment group with three neoliquiritin concentrations showed slightly thickened epidermis, less inflammatory cell infiltration in the dermis, and non-obvious acanthosis, with the treatment group with 1.0 wt % neoliquiritin showing more significant improvement. Furthermore, quantification with ImageJ revealed that the epidermal thickness of the model group was significantly increased compared to the normal control group (^{###}p<0.001).

The epidermal thickness of the treatment group with three neoliquiritin concentrations was significantly decreased compared to the model group (***p<0.001) (FIG. 3B). Meanwhile, the number of dermal lymphocytes in the model group was significantly increased compared to the normal control group (^{###}p<0.001). The number of dermal lymphocytes in the treatment group with three neoliquiritin concentrations was significantly decreased compared to the model group (***p<0.001) (FIG. 3C). The treatment group with 1.0 wt % neoliquiritin performed better than the treatment groups with 0.5 wt % and 1.5 wt % neoliquiritin in terms of both epidermal thickness and dermal lymphocyte count, and was thus selected for further safety studies.

### 5.3. Topical Administration of Neoliquiritin Showing No Effect on the Liver, Spleen, or Kidneys of Calcipotriol-Induced AD Mouse Model

Histopathological sections of the liver, spleen, and kidney tissues of mice in each group (FIG. 4, scale bar=100 μm) showed no significant differences between the normal control group and the treatment group with 1.0 wt % neoliquiritin, demonstrating that neoliquiritin administration did not cause toxic side effects on the liver, spleen, or kidneys.

The TCM monomer, neoliquiritin, significantly alleviated skin erythema, dryness, scratch marks, and epidermal erosion and desquamation in the diseased mice, demonstrating remarkable efficacy and warranting promotion.

The above are merely preferred implementations of the present disclosure. It should be noted that a person of ordinary skill in the art may further make several improvements and modifications without departing from the principle of the present disclosure, but such improvements and modifications should be deemed as falling within the protection scope of the present disclosure.

What is claimed is:

1. A topical formulation for treating atopic dermatitis (AD), wherein an active ingredient of the topical formulation is neoliquiritin, and wherein the neoliquiritin has a concentration of 0.5 wt % to 1.5 wt %.

2. The topical formulation according to claim 1, wherein the neoliquiritin has a concentration of 1 wt %.

3. The topical formulation according to claim 1, wherein a dosage form of the topical formulation is selected from the group consisting of a patch, a paste, an ointment, a cream, a gel, an oil, a microneedle preparation, a coating agent, a cataplasm, a spray, and a dressing.

4. The topical formulation according to claim 3, wherein the topical formulation further comprises a pharmaceutically acceptable auxiliary material.

5. A cream for treating AD, comprising following components by mass concentration: 0.5% to 1.5% of neoliquiritin, 4% to 13% of a solvent, 5% to 25% of an oil phase, 56% to 87% of an aqueous phase, 2% to 8% of an emulsifier, and 0.05% to 0.15% of a preservative.

6. The cream according to claim 5, comprising the following components by mass concentration: 0.8% to 1.2% of the neoliquiritin, 6% to 10% of the solvent, 10% to 20% of the oil phase, 65% to 75% of the aqueous phase, 3% to 5% of the emulsifier, and 0.08% to 0.12% of the preservative.

7. The cream according to claim 5, comprising the following components by mass concentration: 1% of the neoliquiritin, 8.3% of the solvent, 14.0% of the oil phase, 71.9% of the aqueous phase, 4.7% of the emulsifier, and 0.1% of the preservative.

8. The cream according to claim 5, wherein the solvent is one or more selected from the group consisting of diethylene glycol monoethyl ether, ethanol, and propylene glycol.

9. The cream according to claim 5, wherein the oil phase is one or more selected from the group consisting of glyceryl mono- and distearate, cetostearyl alcohol, liquid paraffin, vaseline, and lanolin.

10. The cream according to claim 9, wherein the oil phase is composed of the glyceryl mono- and distearate, the cetostearyl alcohol, and the liquid paraffin at a mass ratio of 3:5:6.

11. The cream according to claim 5, wherein the aqueous phase is one or more selected from the group consisting of purified water, deionized water, and glycerol.

12. The cream according to claim 11, wherein the aqueous phase is composed of the purified water and the glycerol at a mass ratio of 9.3:1.

13. The cream according to claim 5, wherein the emulsifier is one or more selected from the group consisting of polyethylene glycol-7-stearate, polyoxyethylene (21) stearyl ether, polyoxyethylene (2) stearyl ether, polyglyceryl oleate, and sodium lauryl sulfate.

14. The cream according to claim 13, wherein the emulsifier is composed of the polyethylene glycol-7-stearate, the polyoxyethylene (21) stearyl ether, the polyoxyethylene (2) stearyl ether, and the polyglyceryl oleate at a mass ratio of 4:9:9:6.

15. The cream according to claim 5, wherein the preservative is one or more selected from the group consisting of ethylparaben, methylparaben, and phenoxyethanol.

16. A method for preparing the cream according to claim 5, comprising following steps: (1) weighing and mixing the oil phase, the emulsifier, and the preservative to obtain a first mixture, weighing the aqueous phase to obtain a second mixture, and dissolving the neoliquiritin with the solvent to obtain a third mixture; (2) mixing the first mixture and the second mixture under heating, homogenizing, adding the third mixture and homogenizing again; and (3) cooling an obtained twice-homogenized product to obtain the cream.

17. The preparation method according to claim 16, wherein step (2) comprises the following steps: heating the first mixture and the second mixture to 85° C. before the mixing, homogenizing for 3 min, cooling the obtained homogenized product to 60° C., adding with the third mixture, and then homogenizing again for 3 min.

18. A method for treating AD, comprising administering the topical formulation according to claim 1 to a subject in need thereof.

19. A method for inhibiting epidermal proliferation and/or dermal lymphocyte infiltration, comprising administering the topical formulation according to claim 1 to a subject in need thereof.


SEQUENCE LISTING (8 sequences)

SEQ ID NO: 1 - DNA, 22 bp, synthetic construct
  source 1..22: mol_type = other DNA
caccattggcaatgagcggttc

SEQ ID NO: 2 - DNA, 22 bp, synthetic construct
  source 1..22: mol_type = other DNA
aggtctttgcggatgtccacgt

SEQ ID NO: 3 - DNA, 23 bp, synthetic construct
  source 1..23: mol_type = other DNA
ccgtaacagacatctttgctgcc

SEQ ID NO: 4 - DNA, 22 bp, synthetic construct
  source 1..22: mol_type = other DNA
gagtgtccttctcatggtggct

SEQ ID NO: 5 - DNA, 22 bp, synthetic construct
  source 1..22: mol_type = other DNA
acggtcattgctctcacttgcc

SEQ ID NO: 6 - DNA, 23 bp, synthetic construct
  source 1..23: mol_type = other DNA
ctgtcaggttgatgctccatacc

SEQ ID NO: 7 - DNA, 22 bp, synthetic construct
  source 1..22: mol_type = other DNA
tatctggtgcccaggctattcg

SEQ ID NO: 8 - DNA, 22 bp, synthetic construct
  source 1..22: mol_type = other DNA
tgaagcgacgccacaatccttg